(19) United States
(12) Patent Application Publication
(10) Pub. No.: US 2025/0213648 A1
(43) Pub. Date: Jul. 3, 2025

(54) OXYTOCIN COMPOSITIONS AND METHODS

(71) Applicant: Endo Operations Limited, Ballsbridge (IE)

(72) Inventors: Kumaresh Soppimath, Skillman, NJ (US); Mukesh Tiwari, Navi Mumbai (IN); Pramod Sunil Kadam, Navi Mumbai (IN); Dhiraj Kumar Chopra, Navi Mumbai (IN)

(21) Appl. No.: 19/008,443

(22) Filed: Jan. 2, 2025

Related U.S. Application Data
(60) Provisional application No. 63/616,999, filed on Jan. 2, 2024.

(51) Int. Cl. A61K 38/095; A61K 9/08; A61K 47/02; A61K 47/12

(52) U.S. Cl. CPC A61K 38/095; A61K 9/08; A61K 47/02; A61K 47/12

(57) ABSTRACT

Disclosed herein is a stable oxytocin composition having between about 0.04 and 0.08 IU/mL of oxytocin and/or a pharmaceutically acceptable salt thereof, a buffer comprising sodium acetate trihydrate and acetic acid, a tonicity adjusting agent comprising sodium chloride, a pH adjusting agent comprising acetic acid, sodium hydroxide, or a combination thereof, and water, the composition having a total impurities concentration of equal to or less than 16% after storage of at least six months at 25° C. Also disclosed herein is a pharmaceutical product having a single-use container containing the sterile and storage-stable liquid composition of the present disclosure.

### CROSS-REFERENCE TO RELATED APPLICATIONS

This application claims priority to U.S. Provisional Application No. 63/616,999, filed Jan. 2, 2024, the disclosure of which is incorporated herein by reference.

### FIELD OF THE INVENTION

The field of the invention is oxytocin compositions and methods, and particularly as it relates to sterile and storage stable ready-to-use formulations for infusion.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Oxytocin is a natural small peptide hormone comprising nine amino acids and an intramolecular disulfide bond and performs several physiological functions reproduction, childbirth, and postpartum bleeding control. As a pharmaceutical preparation, oxytocin is one of the essential medicines listed by the WHO and is indicated for induction of labor and prevention and treatment of postpartum hemorrhage, one of the leading causes of maternal mortality.

Oxytocin is sensitive to high temperatures and oxidation, and poor manufacturing practices, inappropriate transport and/or storage conditions often lead to significant degradation. The most common degradation mechanisms include deamidation at Gln⁴, Asn⁵, and Gly⁹, oxidation at Tyr^{e}, and dimerization/oxidation as well as tri-/tetrasulfide formation and beta elimination at the intramolecular disulfide bond. Most of the commercially available oxytocin products require storage at 2-8° C., which may be problematic in developing countries and/or tropical or sub-tropical locations. Certain oxytocin formulations are also known that can be stored without the need for refrigeration, however such formulations typically require certain preservatives that may be undesirable.

For example, US 2017/0157201 teaches various stabilizing agents for oxygen sensitive drugs, including oxytocin. However, such stabilizers may affect the action of the active ingredient, and/or may not be well tolerated, especially when administered in larger quantities. In other examples (e.g., US 2019/0388498), a ready-to-use oxytocin formulation is presented that is free of chlorobutanol. However, stability data and information regarding the preparation of such formulations were not presented. In yet another known approach, specific autoclaving conditions with limited air overpressure were used to preserve integrity of pharmaceutically active agents as described, for example, in US 2020/0289439 and US 2014/0303254. However, stability data and information regarding the preparation of oxytocin-containing formulations were not presented.

Thus, even though various compositions and methods for oxytocin formulations are known in the art, all or almost all of them suffer from several drawbacks, particularly where the formulations are in a sterile and storage-stable ready-to-use form. Therefore, there remains a need for compositions and methods for sterile and storage-stable ready-to-use oxytocin formulations.

### SUMMARY OF THE INVENTION

The inventive subject matter is directed to various compositions and methods for sterile and storage stable ready-to-use oxytocin formulations. Advantageously, the formulations have an oxytocin concentration that allows direct infusion without further dilution, thereby simplifying administration and safety of use. Degradation of the oxytocin in contemplated formulations may be less than 10% after storage without the need for refrigeration over at least 6 months, and optionally at least 12 months.

According to some aspects of the disclosure, a sterile and storage-stable liquid composition of oxytocin may include a buffered aqueous carrier comprising oxytocin at a concentration of at least 100 ng/mL, wherein the liquid composition has a pH of between 4.0 and 5.0, and wherein the liquid composition is formulated such that, upon storage of the composition at 25° C. for at least 6 months, at least 90% of the oxytocin in the formulation remains undegraded. The liquid composition may be a filter sterilized composition.

In some embodiments the buffer is an acetate buffer (e.g., to effect a pH of the liquid composition of between 4.5 and 4.8), and/or the aqueous carrier comprises deoxygenated water (e.g., comprising equal or less than 1 ppm of dissolved oxygen). Where desired, the aqueous carrier may further include an antioxidant such as ascorbic acid or alpha-tocopherol. Typically, but not necessarily, the concentration of oxytocin is at least 160 ng/mL, or at least 320 ng/mL, or at least 1,000 ng/mL, or at least 1,600 ng/mL.

In further embodiments, it is preferred that upon storage of the composition at 25° C. for at least 6 months, at least 95% or at least 97% of the oxytocin in the formulation will remain undegraded.

Consequently, the inventors also contemplate a pharmaceutical product that comprises a single-use container containing the sterile and storage-stable liquid composition of oxytocin as presented herein, wherein the single-use container contains the sterile and storage-stable liquid composition in an amount that provides between 10 and 100 IU of oxytocin.

For example, contemplated single-use container especially include an infusion bag or a prefilled syringe. The sterile and storage-stable liquid composition may be a filter-sterilized composition. Where desired, the single-use container may be packaged into a secondary overwrap, and an oxygen scavenger may be disposed in a space between the single-use container and the secondary overwrap.

In one exemplary formulation, a rubber stoppered glass vial with an aluminum seal may contain 125 mL of a filter-sterilized composition that uses as a carrier water for injection, the composition containing 7.5 IU of oxytocin and/or a pharmaceutically acceptable salt thereof, a buffer containing about 0.2 mg/mL sodium acetate trihydrate and about 0.514 mg/mL of acetic acid, about 9.0 mg/mL of sodium chloride as a tonicity adjusting agent, and acetic acid and/or sodium hydroxide as pH adjusting agents suitable to adjust an initial pH range of the composition to 3.0 to 5.0.

In one exemplary formulation, a rubber stoppered glass vial with an aluminum seal may contain 250 mL of a filter-sterilized composition that uses as a carrier water for injection, the composition containing 15 IU of oxytocin and/or a pharmaceutically acceptable salt thereof, a buffer containing about 0.2 mg/mL sodium acetate trihydrate and about 0.514 mg/mL of acetic acid, about 9.0 mg/mL of sodium chloride as a tonicity adjusting agent, and acetic acid and/or sodium hydroxide as pH adjusting agents suitable to adjust an initial pH range of the composition to 3.0 to 5.0.

In one exemplary formulation, a flexible bag for infusion, optionally within an aluminum pouch, may contain 500 mL of a filter-sterilized composition that uses as a carrier water for injection, the composition containing 30 IU of oxytocin and/or a pharmaceutically acceptable salt thereof, a buffer containing about 0.2 mg/mL sodium acetate trihydrate and about 0.514 mg/mL of acetic acid, about 9.0 mg/mL of sodium chloride as a tonicity adjusting agent, and acetic acid and/or sodium hydroxide as pH adjusting agents suitable to adjust an initial pH range of the composition to 3.0 to 5.0.

In one exemplary formulation, a flexible bag for infusion, optionally within an aluminum pouch, may contain 1,000 mL of a filter-sterilized composition that uses as a carrier water for injection, the composition containing 20 IU of oxytocin and/or a pharmaceutically acceptable salt thereof, a buffer containing about 0.2 mg/mL sodium acetate trihydrate and about 0.514 mg/mL of acetic acid, about 9.0 mg/mL of sodium chloride as a tonicity adjusting agent, and acetic acid and/or sodium hydroxide as pH adjusting agents suitable to adjust an initial pH range of the composition to 3.0 to 5.0.

Various objects, features, aspects, and advantages of the inventive subject matter will become more apparent from the following detailed description of preferred embodiments.

### DETAILED DESCRIPTION

The inventors have discovered that sterile and storage-stable oxytocin compositions can be prepared that contain therapeutically effective quantities of oxytocin and/or a pharmaceutically acceptable salt thereof in a ready-to-use format. Advantageously, contemplated compositions will have significant storage stability (e.g., exhibit less than 10% degradation of oxytocin) over even extended periods of time without the need for refrigeration and can be used for direct administration without the need for dilution.

In some non-limiting examples, the composition may include a carrier, one or more tonicity adjusting agents, one or more buffers, one or more pH adjusting agents, and oxytocin and/or a pharmaceutically acceptable salt thereof.

In one exemplary formulation, a flexible bag for infusion may contain 100 mL of a filter-sterilized composition that uses as a carrier water for injection buffered with 50 mM acetate buffer at a pH of 4.8 and in which the 100 mL of the composition contain 10 IU of oxytocin and/or a pharmaceutically acceptable salt thereof.

In one exemplary formulation, a rubber stoppered glass vial with an aluminum seal may contain 125 mL of a filter-sterilized composition that uses as a carrier water for injection, the composition containing 7.5 IU of oxytocin and/or a pharmaceutically acceptable salt thereof, a buffer containing about 0.2 mg/mL sodium acetate trihydrate and about 0.514 mg/mL of acetic acid, about 9.0 mg/mL of sodium chloride as a tonicity adjusting agent, and acetic acid and/or sodium hydroxide as pH adjusting agents suitable to adjust an initial pH range of the composition to 3.0 to 5.0.

In one exemplary formulation, a rubber stoppered glass vial with an aluminum seal may contain 250 mL of a filter-sterilized composition that uses as a carrier water for injection, the composition containing 15 IU of oxytocin and/or a pharmaceutically acceptable salt thereof, a buffer containing about 0.2 mg/mL sodium acetate trihydrate and about 0.514 mg/mL of acetic acid, about 9.0 mg/mL of sodium chloride as a tonicity adjusting agent, and acetic acid and/or sodium hydroxide as pH adjusting agents suitable to adjust an initial pH range of the composition to 3.0 to 5.0.

In one exemplary formulation a flexible bag for infusion, optionally within an aluminum pouch, may contain 500 mL of a filter-sterilized composition that uses as a carrier water for injection, the composition containing 30 IU of oxytocin and/or a pharmaceutically acceptable salt thereof, a buffer containing about 0.2 mg/mL sodium acetate trihydrate and about 0.514 mg/mL of acetic acid, about 9.0 mg/mL of sodium chloride as a tonicity adjusting agent, and acetic acid and/or sodium hydroxide as pH adjusting agents suitable to adjust an initial pH range of the composition to 3.0 to 5.0.

In one exemplary formulation, a flexible bag for infusion, optionally within an aluminum pouch, may contain 1,000 mL of a filter-sterilized composition that uses as a carrier water for injection, the composition containing 20 IU of oxytocin and/or a pharmaceutically acceptable salt thereof, a buffer containing about 0.2 mg/mL sodium acetate trihydrate and about 0.514 mg/mL of acetic acid, about 9.0 mg/mL of sodium chloride as a tonicity adjusting agent, and acetic acid and/or sodium hydroxide as pH adjusting agents suitable to adjust an initial pH range of the composition to 3.0 to 5.0.

As will be readily appreciated, contemplated formulations need not be limited to 10 IU per 100 mL, and it is generally contemplated that the compositions presented herein may contain oxytocin and/or a pharmaceutically acceptable salt thereof at a concentration of at least 10 ng/mL, at least 25 ng/mL, at least 50 ng/mL, at least 75 ng/mL, at least 100 ng/mL, at least 200 ng/mL, 500 ng/mL, at least 750 ng/mL, at least 1,000 ng/mL, at least 1,500 ng/mL, and even higher. In some non-limiting examples, the oxytocin and/or pharmaceutically acceptable salt thereof may be present in a concentration of between about 0.001 and 1 IU/mL, optionally between about 0.01 and 0.1 IU/mL, optionally between about 0.04 and 0.08 IU/mL, and optionally about 0.06 IU/mL. In some non-limiting examples, the oxytocin and/or pharmaceutically acceptable salt thereof may be present in a concentration of between about 0.001 and 1 IU/mL, optionally between about 0.001 and 0.1 IU/mL, optionally between about 0.01 and 0.07 IU/mL, optionally between about 0.01 and 0.03 IU/mL, and optionally about 0.02 IU/mL. Viewed from a different perspective, it is generally preferred that the oxytocin concentration of contemplated compositions will be suitable for direct infusion (i.e., infusion without the need for dilution) and as such will provided in a container that will contain therapeutically effective quantities of oxytocin and/or a pharmaceutically acceptable salt thereof.

In this context, it should be noted that 1 mg of oxytocin may be equivalent to 600 IU of biological activity (see WHO Working document QAS/07.241/FINAL of June 2010). Per the US Pharmacopeia (USP), its oxytocin activity is not less than 400 USP Oxytocin Units per mg. Therefore, contemplated compositions may be provided as ready-to-use compositions in which a single-use container contains at least 1 IU of oxytocin and/or a pharmaceutically acceptable salt thereof, or at least 5 IU of oxytocin and/or a pharmaceutically acceptable salt thereof, or at least 10 IU of oxytocin and/or a pharmaceutically acceptable salt thereof, or at least 20 IU of oxytocin and/or a pharmaceutically acceptable salt thereof, or at least 40 IU of oxytocin and/or a pharmaceutically acceptable salt thereof, or at least 60 IU of oxytocin and/or a pharmaceutically acceptable salt thereof, or at least 80 IU of oxytocin and/or a pharmaceutically acceptable salt thereof, or at least 100 IU of oxytocin and/or a pharmaceutically acceptable salt thereof, or even more. In some non-limiting examples, the composition may include between about 1 and 40 IU of oxytocin and/or a pharmaceutically acceptable salt thereof, optionally between about 7.5 and 30 IU of oxytocin and/or a pharmaceutically acceptable salt thereof. In some non-limiting examples, the composition may include about 5 IU of oxytocin and/or a pharmaceutically acceptable salt thereof, optionally about 5.5 IU, optionally about 6 IU, optionally about 6.5 IU, optionally about 7 IU, optionally about 7.5 IU, optionally about 8 IU, optionally about 8.5 IU, optionally about 9 IU, optionally about 9.5 IU, optionally about 10 IU, optionally about 10.5 IU, optionally about 11 IU, optionally about 11.5 IU, optionally about 12 IU, optionally about 12.5 IU, optionally about 13 IU, optionally about 13.5 IU, optionally about 14 IU, optionally about 14.5 IU, optionally about 15 IU of oxytocin, optionally about 15.5 IU, optionally about 16 IU, optionally about 16.5 IU, optionally about 17 IU, optionally about 17.5 IU, optionally about 18 IU, optionally about 18.5 IU, optionally about 19 IU, optionally about 19.5 IU, optionally about 20 IU, optionally about 20.5 IU, optionally about 21 IU, optionally about 21.5 IU, optionally about 22 IU, optionally about 22.5 IU, optionally about 23 IU, optionally about 23.5 IU, optionally about 24 IU, optionally about 24.5 IU, optionally about 25 IU of oxytocin, optionally about 25.5 IU, optionally about 26 IU, optionally about 26.5 IU, optionally about 27 IU, optionally about 27.5 IU, optionally about 28 IU, optionally about 28.5 IU, optionally about 29 IU, optionally about 29.5 IU, and optionally about 30 IU.

The volume of the compositions presented herein may be between about 1 mL and 1,000 mL, such as volumes of at least 1 mL, or at least 5 mL, or at least 10 mL, or at least 20 mL, or at least 50 mL, or at least 100 mL, or at least 200 mL, or at least 250 mL, or at least 500 mL, or even more. In some non-limiting examples, the composition may have a volume of between about 50 and 600 mL, optionally between about 100 and 200 mL, optionally between about 200 and 300 mL, optionally between about 300 and 400 mL, optionally between about 400 and 500 mL, and optionally between about 450 and 550 mL. In some non-limiting examples, the composition may have a volume of about 100 mL, optionally about 150 mL, optionally about 200 mL, optionally about 250 mL, optionally about 300 mL, optionally about 350 mL, optionally about 400 mL, optionally about 450 mL, optionally about 500 mL, optionally about 550 mL, optionally about 600 mL, optionally about 650 mL, optionally about 700 mL, optionally about 750 mL, optionally about 800 mL, optionally about 850 mL, optionally about 900 mL, optionally about 950 mL, and optionally about 1,000 mL.

Still further, it should be noted that the term “oxytocin” as used herein includes the unmodified human peptide form of oxytocin (see e.g., UniProtKB—P01178), as well as its functional analogs, such as for example, desamino-oxytocin, carbetocin, 4-threonine-1-hydroxy-deaminooxytocin, 9-deamidooxytocin, 7-D-proline-oxytocin and its deamino analog, (2,4-diisoleucine)-oxytocin, 1-desamino-1-monocarba-E12-Tyr (OMe)]-OT(dCOMOT), [Thr⁴-Glyl-oxytocin (TG-OT), oxytocin agonists as described by Olson et al., (Peptides 12(1): 113-118, 1991), oxypressin, and deamino-6-carba-oxytoxin (dC60). Further examples of oxytocin analogues (oxytocics) can for instance be found in MX 9707899, including the oxytocin analogues (Mpa¹) oxytocin, (D-Asn⁵) oxytocin, (D¹-Cys¹) oxytocin, (Gly⁴) oxytocin, (Leu⁴, D-Arg⁸) oxytocin, (Mpa¹, Leu⁴, D-Arg⁸) oxytocin, (Arg⁸) oxytocin and (Ile′) oxytocin.

Non-limiting examples of pharmaceutically acceptable salts include hydrochloride salts, sodium salts, sulfate salts, acetate salts, phosphate salts, diphosphate salts, chloride salts, potassium salts, maleate salts, calcium salts, citrate salts, mesylate salts, nitrate salts, tartrate salts, aluminum salts, gluconate salts, and combinations thereof. According to some aspects, the composition includes oxytocin acetate.

With respect to suitable carriers, it is generally contemplated that the predominant (or even exclusive) component may be water, which may be (but is not limited to) deoxygenated water. In some non-limiting examples, the carrier according to the present disclosure may include water for injection (WFI). As used herein, the term “water for injection” or “WFI” refers to sterile, non-pyrogenic, distilled water suitable for intravenous administration after addition of a suitable solute. According to some aspects, WFI may refer to water that meets USP requirements for WFI. For example, WFI may refer to water that includes less than 0.25 USP Endotoxin Unit/mL per the Bacterial Endotoxins Test <85>, meets the requirements as set forth in Water Conductivity, Bulk Water <645>, and meets the requirements as set forth in Total Organic Carbon <643>.

However, in further contemplated aspects, one or more cosolvents are also deemed suitable for use herein, and all pharmaceutically acceptable cosolvents are deemed appropriate for use herein. Most typically, but not necessarily, the water/solvent will be sparged with an inert gas (e.g., N₂, Ar, Ne, He, etc.) for a time sufficient to reduce dissolved oxygen. Thus, it is contemplated that the liquid carrier may have a residual dissolved oxygen content of equal or less than 2 ppm, or equal or less than 1 ppm, or equal or less than 0.5 ppm.

It is still further contemplated that the oxytocin compositions presented herein may include a buffer that stabilizes the pH of the contemplated liquid compositions in an acidic pH range. According to some aspects, the buffer may be a buffer system. As used herein, the term “buffer system” refers to a component of a composition that provides a resistance to significant change in pH caused by a strong acid or base. A buffer system may include two or more buffer components, such as a weak acid and its conjugate base. A buffer system may provide a resistance to a significant pH change by interacting with a strong acid or strong base in a composition or solution, thereby at least partially preventing the pH of the composition or solution from changing significantly.

Suitable buffers include, but are not limited to, glycine buffers, citrate buffers, citrate/phosphate buffers, acetate buffers, etc. In some non-limiting examples, the one or more buffer components may include the acids or salt forms of one or more of lactate, tartarate, glutamate, malate, citrate, gluconate, benzoate, succinate, acetate, glycine, and aspartate. Additionally or alternatively, the one or more buffer components may include lithium hydroxide, sodium hydroxide, potassium hydroxide, rubidium hydroxide, cesium hydroxide, calcium hydroxide, strontium hydroxide, sodium phosphate dibasic dihydrate, sodium acetate trihydrate, and/or barium hydroxide. In some non-limiting aspects, the buffer components may include sodium acetate trihydrate, acetic acid, or a combination thereof.

According to some aspects, the buffer may be provided sufficient to stabilizes the pH of the composition within a certain pH range. According to some aspects, the pH range may be less than 5.5, optionally less than 5.0, such as for example, less than 4.7, or less than 4.5, but higher than 3.0, optionally higher than 3.5, and optionally higher than 3.7 or 4.0. Additionally or alternatively, the pH range may be between about 2.5 and 5.5, optionally between about 3.0 and 5.0, optionally between about 3.7 and 5.3, optionally between 4.0 and 5.0, optionally between 4.8 and 4.2, optionally between 4.7 and 4.4, and optionally between 4.5 and 4.6. Additionally or alternatively, the pH range may be between about 2.0 and 6.0, optionally between about 3.0 and 5.0, optionally between about 3.1 and 4.9, optionally between 3.2 and 4.8, optionally between 3.3 and 4.7, optionally between 3.4 and 4.6, optionally between 3.5 and 4.5, optionally between about 3.6 and 4.4, optionally between about 3.7 and 4.3, optionally between about 3.8 and 4.2, optionally between about 3.9 and 4.1, and optionally about 4.0. Notably, it is contemplated that the pH range may provide remarkable stability for low concentrations of oxytocin, alone or in combination with one or more of an antioxidant, a chelator, and/or a tonicity agent. While not limiting to the inventive subject matter, the buffer strength may be relatively low, for example, equal or less than 100 mM, and more typically equal or less than 50 mM, and most typically between 5 mM and 20 mM (e.g., 10 mM).

According to some aspects, each of the one or more buffer components may be present in the composition at a concentration in the range of between about 0.01 and 5 mg/mL, optionally between about 0.01 and 2 mg/mL, optionally between about 0.01 and 1 mg/mL, optionally between about 0.1 and 0.6 mg/mL, optionally between about 0.1 and 0.3 mg/mL, and optionally about 0.2 mg/mL.

According to some aspects, each of the one or more buffer components may be present in the composition at a concentration in the range of between about 0.01 and 5 mg/mL, optionally between about 0.01 and 2 mg/mL, optionally between about 0.01 and 1 mg/mL, optionally between about 0.1 and 0.6 mg/mL, optionally between about 0.4 and 0.6 mg/mL, optionally about 0.5 mg/mL, and optionally about 0.514 mg/mL.

According to some aspects, a total concentration of buffer components in the composition may be in the composition at a concentration in the range of between about 0.01 and 10 mg/mL, optionally between about 0.01 and 5 mg/mL, optionally between about 0.01 and 1 mg/mL, optionally between about 0.5 and 0.9 mg/mL, optionally about 0.7 mg/mL, and optionally about 0.714 mg/mL.

The one or more buffer components may be present at a concentration that provides a resistance to a significant change in the composition's pH after a certain period of shelf life. As used herein, a significant change in pH may be more than 5 pH units, optionally more than 4.5 pH units, optionally more than 4 pH units, optionally more than 3.5 pH units, optionally more than 3 pH units, optionally more than 2.5 pH units, optionally more than 2 pH units, optionally more than 1.5 pH units, optionally more than 1 pH units, optionally more than 0.5 pH units, optionally more than 0.05 pH units, and optionally more than 0.01 pH units.

The period of shelf life of the composition may be 1 month, preferably 2 months, more preferably 3 months, more preferably 4 months, more preferably 5 months, more preferably 6 months, more preferably 7 months, more preferably 8 months, more preferably 9 months, more preferably 10 months, more preferably 11 months, more 12 months, preferably 13 months, more preferably 14 months, more preferably 15 months, more preferably 16 months, more preferably 17 months, more preferably 18 months, more preferably 19 months, more preferably 20 months, more preferably 21 months, more preferably 22 months, more preferably 23 months, more preferably 24 months, more preferably 25 months, more preferably 26 months, more preferably 27 months, more preferably 28 months, more preferably 29 months, more preferably 30 months, more preferably 31 months, more preferably 32 months, more preferably 33 months, more preferably 34 months, more preferably 35 months, and most preferably 36 months. According to some aspects, the period of shelf life may vary based on product presentation.

According to some aspects, shelf life may be determined by measuring certain characteristics of the composition that may indicate that the composition is unfit for medical use. According to some aspects, shelf life may be determined by measuring the concentration of impurities in the composition after storage at 5° C.±3° C. According to some aspects, shelf life may be determined by measuring the concentration of impurities in the composition after storage at 25° C.±5° C. According to some aspects, shelf life may be determined by measuring the concentration of impurities in the composition after storage at 40° C.±5° C. According to some aspects, shelf life may be determined by measuring the concentration of impurities in the composition after storage at 60° C.±5° C. According to some aspects, shelf life may be determined by assaying the amount of the active ingredient (e.g., the oxytocin and/or pharmaceutically acceptable salt thereof) still present in its unaltered form.

According to some aspects, the oxytocin compositions may further include one or more pH adjusting agents suitable to adjust an initial pH range of the composition. According to some aspects, the initial pH range may be less than 5.5, optionally less than 5.0, such as for example, less than 4.7, or less than 4.5, but higher than 3.0, optionally higher than 3.5, and optionally higher than 3.7 or 4.0. Additionally or alternatively, the initial pH range may be between about 2.5 and 5.5, optionally between about 3.0 and 5.0, optionally between about 3.7 and 5.3, optionally between 4.0 and 5.0, optionally between 4.8 and 4.2, optionally between 4.7 and 4.4, and optionally between 4.5 and 4.6. Additionally or alternatively, the initial pH range may be between about 2.0 and 6.0, optionally between about 3.0 and 5.0, optionally between about 3.1 and 4.9, optionally between 3.2 and 4.8, optionally between 3.3 and 4.7, optionally between 3.4 and 4.6, optionally between 3.5 and 4.5, optionally between about 3.6 and 4.4, optionally between about 3.7 and 4.3, optionally between about 3.8 and 4.2, optionally between about 3.9 and 4.1, and optionally about 4.0.

In some non-limiting examples, the one or more pH adjusting agents may include the acids or salt forms of one or more of lactate, tartarate, glutamate, malate, citrate, gluconate, benzoate, succinate, acetate, glycine, and aspartate. Additionally or alternatively, the one or more pH adjusting agents may include lithium hydroxide, sodium hydroxide, potassium hydroxide, rubidium hydroxide, cesium hydroxide, calcium hydroxide, strontium hydroxide, sodium phosphate dibasic dihydrate, sodium acetate trihydrate, and/or barium hydroxide. In some non-limiting aspects, the one or more pH adjusting agents may include acetic acid, sodium hydroxide, or a combination thereof.

In still further contemplated aspects, the formulation may optionally include one or more chelating agents, and particularly metal ion chelators. For example, suitable chelators include various bicarboxylic acids, tricarboxylic acids, and aminopolycarboxylic acids such as ethylenediaminetetraacetic acid (EDTA), ethylene glycol-bis((3-aminoethyl ether)-N,N,N′,N′-tetraacetic acid (EGTA), and penta(carboxymethyl)diethylenetriamine (DTPA), and salts and hydrates thereof. Similarly, it is contemplated that the liquid compositions presented herein may optionally include one or more antioxidants, and especially pharmaceutically acceptable antioxidants suitable for injection. In some embodiments, the antioxidant will be a non-thiol/sulfur antioxidant such as ascorbic acid or alpha-tocopherol, while in other embodiments the antioxidant may be sulfite, bisulfite, glutathione, N-acetyl cysteine, etc.

According to some aspects, the composition may include at least one tonicity adjusting agent. The term “tonicity” refers to the effective osmotic pressure equivalent of a solution or composition. According to some aspects, the tonicity adjusting agent may be present in the composition at a concentration sufficient to maintain the osmolality of the composition in a physiologically acceptable range, such as between about 260 to 340 mOsm/kg. An osmometer can be used to check and adjust the amount of tonicity adjusting agent to be added to obtain the desired osmolality.

According to some aspects, the tonicity adjusting agent may be provided in the composition at a concentration of between about 1 and 20 mg/mL, optionally between about 5 and 15 mg/mL, optionally between about 7 and 11 mg/mL, optionally between about 8 and 10 mg/mL, optionally between about 8.5 and 9.5 mg/mL, and optionally about 9 mg/mL.

According to some aspects, the tonicity adjusting agent may include one or more pharmaceutically acceptable salts, such as sodium bromide, sodium chloride, sodium iodide, or a combination thereof. For example, the pharmaceutically acceptable salt may be NaCl, at a concentration of at least 0.6 wt %, or at least 0.7 wt %, or at least 0.8 wt %, or at least 0.9 wt %. Thus, suitable salt concentrations are between 0.6 wt % and 1.2 wt %. Additional or alternative tonicity agents may also be added, and suitable tonicity agents include glycerol, thioglycerol, mannitol, lactose, dextrose, glucose, hydroxypropyl methyl cellulose, polysorbate, propylene glycol, sodium bromide, sodium iodide, sorbitol, urea, xylitol, and combinations thereof.

Regardless of the specific composition, it is preferred that the liquid compositions will be sterilized in a manner other than autoclaving. Therefore, all manners of aseptic filling sterile containers are deemed suitable for use herein, and particularly contemplated manners of sterilization include sterile filtration, gamma- and/or e-beam sterilization, sterilization with various gases, etc. In some non-limiting examples, sterilization may include filtration through a sterile membrane filter, such as a 0.2 m filter.

Once sterilized, it is generally preferred that the liquid composition is filled into a container, such as a single-use container or a multi-use container. Non-limiting examples of single-use containers include flexible polymeric bags (including, but not limited to, those having a volume of between 50 mL and 1,000 mL) and syringes (including, but not limited to, those having a volume of between 1 mL and 50 mL, or between 1 mL and 500 mL, or between 125 mL and 500 mL). In some non-limiting examples, the container may include a sealable vial. According to some aspects, the container may include metal, glass, rubber, plastic, or a combination thereof, such as a glass vial with a rubber stopper, optionally provided with a seal (e.g., an aluminum seal). Furthermore, it is contemplated that the single-use or multi-use container may be further packed into a secondary overwrap, such as an aluminum pouch or other single- or multi-layer overwraps. In some non-limiting examples, an oxygen scavenger may be included in the space between the single-use or multi-use container and the secondary overwrap. In some non-limiting examples, the single-use or multi-use container and/or the secondary overwrap may have an oxygen transmission rate (OTR) between 0.0005 to 0.005, or between 0.005 to 0.05, or between 0.05 to 0.5, or between 0.5 to 2.00, or between 1.0 to 5.00 cc/100 in2/24 hrs.

Thusly formulated liquid compositions will have remarkable stability over extended periods. For example, it is contemplated that the below stability of the formulations can be observed upon storage at temperatures of 2-8° C., or 25° C., or 40° C., or 60° C., upon storage of at least 1 month, or at least 2 months, or at least 3 months, or at least 6 months, or at least 12 months, or at least 24 months, and even longer. Notably, stability will be such that degradation of the oxytocin and/or pharmaceutically acceptable salt thereof may be limited to maintain at least 90%, or at least 92%, or at least 94%, or at least 96%, or at least 97%, or at least 98%, or at least 99% of the initial oxytocin amount.

According to some aspects, the composition may have an acceptable initial impurity level and/or an acceptable impurity level after a certain period of shelf life as described herein. As used herein, the term “impurity” refers to an undesired substance in a composition. It should be understood that one or more impurities may be present in an initial composition and/or may be formed after a certain period of shelf life of a composition. For example, one or more impurities may be formed via degradation of one or more components of the composition, such as the oxytocin and/or pharmaceutically acceptable salt thereof. Sources of degradation include, but are not limited to, chelation with metal ions, oxidation, racemization, visible light, ultraviolet light, moisture, heat (including heat from a sterilization process), changes in pH, and composition component interactions. Non-limiting examples of impurities according to aspects of the present disclosure include Des-Gly9-Oxytocin, Asp-5-Oxytocin, Glu-4-Oxytocin, oxytocin trisulfide, N-acetyloxytocin, and other unspecified impurities.

According to some aspects, the composition may have no more than about 20% of total impurities after a certain period of shelf life, preferably no more than about 19.5%, more preferably no more than about 19%, more preferably no more than about 18.5%, preferably no more than about 18%, more preferably no more than about 17.5%, more preferably no more than about 17%, more preferably no more than about 16.5%, more preferably no more than about 16%, more preferably no more than about 15.5%, more preferably no more than about 15%, more preferably no more than about 14.5%, more preferably no more than about 14%, more preferably no more than about 13.5%, more preferably no more than about 13%, more preferably no more than about 12.5%, more preferably no more than about 12%, more preferably no more than about 11.5%, more preferably no more than about 11%, more preferably no more than about 10.5%, more preferably no more than about 10%, more preferably no more than about 9.5%, more preferably no more than about 9%, more preferably no more than about 8.5%, more preferably no more than about 8%, more preferably no more than about 7.5%, more preferably no more than about 7%, more preferably no more than about 6.5%, more preferably no more than about 6%, more preferably no more than about 5.5%, and most preferably no more than about 5%.

According to some aspects, the composition may have no more than about 20% of total impurities after a certain period of shelf life, preferably no more than about 19.9%, more preferably no more than about 19.8%, more preferably no more than about 19.7%, more preferably no more than about 19.6%, more preferably no more than about 19.5%, more preferably no more than about 19.4%, more preferably no more than about 19.3%, more preferably no more than about 19.2%, more preferably no more than about 19.1%, more preferably no more than about 19%, more preferably no more than about 18.9%, more preferably no more than about 18.8%, more preferably no more than about 18.7%, more preferably no more than about 18.6%, more preferably no more than about 18.5%, more preferably no more than about 18.4%, more preferably no more than about 18.3%, more preferably no more than about 18.2%, more preferably no more than about 18.1%, more preferably no more than about 18%, more preferably no more than about 17.9%, more preferably no more than about 17.8%, more preferably no more than about 17.7%, more preferably no more than about 17.6%, more preferably no more than about 17.5%, more preferably no more than about 17.4%, more preferably no more than about 17.3%, more preferably no more than about 17.2%, more preferably no more than about 17.1%, more preferably no more than about 17%, more preferably no more than about 16.9%, more preferably no more than about 16.8%, more preferably no more than about 16.7%, more preferably no more than about 16.6%, more preferably no more than about 16.5%, more preferably no more than about 16.4%, more preferably no more than about 16.3%, more preferably no more than about 16.2%, more preferably no more than about 16.1%, more preferably no more than about 16%, more preferably no more than about 15.9%, more preferably no more than about 15.8%, more preferably no more than about 15.7%, more preferably no more than about 15.6%, more preferably no more than about 15.5%, more preferably no more than about 15.4%, more preferably no more than about 15.3%, more preferably no more than about 15.2%, more preferably no more than about 15.1%, more preferably no more than about 15%, more preferably no more than about 14.9%, more preferably no more than about 14.8%, more preferably no more than about 14.7%, more preferably no more than about 14.6%, more preferably no more than about 14.5%, more preferably no more than about 14.4%, more preferably no more than about 14.3%, more preferably no more than about 14.2%, more preferably no more than about 14.1%, more preferably no more than about 14%, more preferably no more than about 13.9%, more preferably no more than about 13.8%, more preferably no more than about 13.7%, more preferably no more than about 13.6%, more preferably no more than about 13.5%, more preferably no more than about 13.4%, more preferably no more than about 13.3%, more preferably no more than about 13.2%, more preferably no more than about 13.1%, more preferably no more than about 13%, more preferably no more than about 12.9%, more preferably no more than about 12.8%, more preferably no more than about 12.7%, more preferably no more than about 12.6%, more preferably no more than about 12.5%, more preferably no more than about 12.4%, more preferably no more than about 12.3%, more preferably no more than about 12.2%, more preferably no more than about 12.1%, more preferably no more than about 12%, more preferably no more than about 11.9%, more preferably no more than about 11.8%, more preferably no more than about 11.7%, more preferably no more than about 11.6%, more preferably no more than about 11.5%, more preferably no more than about 11.4%, more preferably no more than about 11.3%, more preferably no more than about 11.2%, more preferably no more than about 11.1%, more preferably no more than about 11%, more preferably no more than about 10.9%, more preferably no more than about 10.8%, more preferably no more than about 10.7%, more preferably no more than about 10.6%, more preferably no more than about 10.5%, more preferably no more than about 10.4%, more preferably no more than about 10.3%, more preferably no more than about 10.2%, more preferably no more than about 10.1%, more preferably no more than about 10%, more preferably no more than about 9.9%, more preferably no more than about 9.8%, more preferably no more than about 9.7%, more preferably no more than about 9.6%, more preferably no more than about 9.5%, more preferably no more than about 9.4%, more preferably no more than about 9.3%, more preferably no more than about 9.2%, more preferably no more than about 9.1%, more preferably no more than about 9%, more preferably no more than about 8.9%, more preferably no more than about 8.8%, more preferably no more than about 8.7%, more preferably no more than about 8.6%, more preferably no more than about 8.5%, more preferably no more than about 8.4%, more preferably no more than about 8.3%, more preferably no more than about 8.2%, more preferably no more than about 8.1%, more preferably no more than about 8%, more preferably no more than about 7.9%, more preferably no more than about 7.8%, more preferably no more than about 7.7%, more preferably no more than about 7.6%, more preferably no more than about 7.5%, more preferably no more than about 7.4%, more preferably no more than about 7.3%, more preferably no more than about 7.2%, more preferably no more than about 7.1%, more preferably no more than about 7%, more preferably no more than about 6.9%, more preferably no more than about 6.8%, more preferably no more than about 6.7%, more preferably no more than about 6.6%, more preferably no more than about 6.5%, more preferably no more than about 6.4%, more preferably no more than about 6.3%, more preferably no more than about 6.2%, more preferably no more than about 6.1%, more preferably no more than about 6%, more preferably no more than about 5.9%, more preferably no more than about 5.8%, more preferably no more than about 5.7%, more preferably no more than about 5.6%, more preferably no more than about 5.5%, more preferably no more than about 5.4%, more preferably no more than about 5.3%, more preferably no more than about 5.2%, more preferably no more than about 5.1%, more preferably no more than about 5%, more preferably no more than about 4.9%, more preferably no more than about 4.8%, more preferably no more than about 4.7%, more preferably no more than about 4.6%, more preferably no more than about 4.5%, more preferably no more than about 4.4%, more preferably no more than about 4.3%, more preferably no more than about 4.2%, more preferably no more than about 4.1%, more preferably no more than about 4%, more preferably no more than about 3.9%, more preferably no more than about 3.8%, more preferably no more than about 3.7%, more preferably no more than about 3.6%, more preferably no more than about 3.5%, more preferably no more than about 3.4%, more preferably no more than about 3.3%, more preferably no more than about 3.2%, more preferably no more than about 3.1%, more preferably no more than about 3%, more preferably no more than about 2.9%, more preferably no more than about 2.8%, more preferably no more than about 2.7%, more preferably no more than about 2.6%, more preferably no more than about 2.5%, more preferably no more than about 2.4%, more preferably no more than about 2.3%, more preferably no more than about 2.2%, more preferably no more than about 2.1%, more preferably no more than about 2%, more preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, and most preferably about 0%.

In some non-limiting examples, the composition may have a total impurity concentration of no more than between about 15 and 17% after a certain period of shelf life, optionally no more than between about 15.1 and 16.9%, optionally no more than between about 15.2 and 16.8%, optionally no more than between about 15.3 and 16.7%, optionally no more than between about 15.4 and 16.6%, optionally no more than between about 15.5 and 16.5%, optionally no more than between about 15.6 and 16.4%, optionally no more than between about 15.7 and 16.3%, optionally no more than between about 15.8 and 16.1%, and optionally no more than between about 15.9 and 16.1%.

According to some aspects, a concentration of each impurity described herein may independently be no more than about 20% after a certain period of shelf life, preferably no more than about 19.9%, more preferably no more than about 19.8%, more preferably no more than about 19.7%, more preferably no more than about 19.6%, more preferably no more than about 19.5%, more preferably no more than about 19.4%, more preferably no more than about 19.3%, more preferably no more than about 19.2%, more preferably no more than about 19.1%, more preferably no more than about 19%, more preferably no more than about 18.9%, more preferably no more than about 18.8%, more preferably no more than about 18.7%, more preferably no more than about 18.6%, more preferably no more than about 18.5%, more preferably no more than about 18.4%, more preferably no more than about 18.3%, more preferably no more than about 18.2%, more preferably no more than about 18.1%, more preferably no more than about 18%, more preferably no more than about 17.9%, more preferably no more than about 17.8%, more preferably no more than about 17.7%, more preferably no more than about 17.6%, more preferably no more than about 17.5%, more preferably no more than about 17.4%, more preferably no more than about 17.3%, more preferably no more than about 17.2%, more preferably no more than about 17.1%, more preferably no more than about 17%, more preferably no more than about 16.9%, more preferably no more than about 16.8%, more preferably no more than about 16.7%, more preferably no more than about 16.6%, more preferably no more than about 16.5%, more preferably no more than about 16.4%, more preferably no more than about 16.3%, more preferably no more than about 16.2%, more preferably no more than about 16.1%, more preferably no more than about 16%, more preferably no more than about 15.9%, more preferably no more than about 15.8%, more preferably no more than about 15.7%, more preferably no more than about 15.6%, more preferably no more than about 15.5%, more preferably no more than about 15.4%, more preferably no more than about 15.3%, more preferably no more than about 15.2%, more preferably no more than about 15.1%, more preferably no more than about 15%, more preferably no more than about 14.9%, more preferably no more than about 14.8%, more preferably no more than about 14.7%, more preferably no more than about 14.6%, more preferably no more than about 14.5%, more preferably no more than about 14.4%, more preferably no more than about 14.3%, more preferably no more than about 14.2%, more preferably no more than about 14.1%, more preferably no more than about 14%, more preferably no more than about 13.9%, more preferably no more than about 13.8%, more preferably no more than about 13.7%, more preferably no more than about 13.6%, more preferably no more than about 13.5%, more preferably no more than about 13.4%, more preferably no more than about 13.3%, more preferably no more than about 13.2%, more preferably no more than about 13.1%, more preferably no more than about 13%, more preferably no more than about 12.9%, more preferably no more than about 12.8%, more preferably no more than about 12.7%, more preferably no more than about 12.6%, more preferably no more than about 12.5%, more preferably no more than about 12.4%, more preferably no more than about 12.3%, more preferably no more than about 12.2%, more preferably no more than about 12.1%, more preferably no more than about 12%, more preferably no more than about 11.9%, more preferably no more than about 11.8%, more preferably no more than about 11.7%, more preferably no more than about 11.6%, more preferably no more than about 11.5%, more preferably no more than about 11.4%, more preferably no more than about 11.3%, more preferably no more than about 11.2%, more preferably no more than about 11.1%, more preferably no more than about 11%, more preferably no more than about 10.9%, more preferably no more than about 10.8%, more preferably no more than about 10.7%, more preferably no more than about 10.6%, more preferably no more than about 10.5%, more preferably no more than about 10.4%, more preferably no more than about 10.3%, more preferably no more than about 10.2%, more preferably no more than about 10.1%, more preferably no more than about 10%, more preferably no more than about 9.9%, more preferably no more than about 9.8%, more preferably no more than about 9.7%, more preferably no more than about 9.6%, more preferably no more than about 9.5%, more preferably no more than about 9.4%, more preferably no more than about 9.3%, more preferably no more than about 9.2%, more preferably no more than about 9.1%, more preferably no more than about 9%, more preferably no more than about 8.9%, more preferably no more than about 8.8%, more preferably no more than about 8.7%, more preferably no more than about 8.6%, more preferably no more than about 8.5%, more preferably no more than about 8.4%, more preferably no more than about 8.3%, more preferably no more than about 8.2%, more preferably no more than about 8.1%, more preferably no more than about 8%, more preferably no more than about 7.9%, more preferably no more than about 7.8%, more preferably no more than about 7.7%, more preferably no more than about 7.6%, more preferably no more than about 7.5%, more preferably no more than about 7.4%, more preferably no more than about 7.3%, more preferably no more than about 7.2%, more preferably no more than about 7.1%, more preferably no more than about 7%, more preferably no more than about 6.9%, more preferably no more than about 6.8%, more preferably no more than about 6.7%, more preferably no more than about 6.6%, more preferably no more than about 6.5%, more preferably no more than about 6.4%, more preferably no more than about 6.3%, more preferably no more than about 6.2%, more preferably no more than about 6.1%, more preferably no more than about 6%, more preferably no more than about 5.9%, more preferably no more than about 5.8%, more preferably no more than about 5.7%, more preferably no more than about 5.6%, more preferably no more than about 5.5%, more preferably no more than about 5.4%, more preferably no more than about 5.3%, more preferably no more than about 5.2%, more preferably no more than about 5.1%, more preferably no more than about 5%, more preferably no more than about 4.9%, more preferably no more than about 4.8%, more preferably no more than about 4.7%, more preferably no more than about 4.6%, more preferably no more than about 4.5%, more preferably no more than about 4.4%, more preferably no more than about 4.3%, more preferably no more than about 4.2%, more preferably no more than about 4.1%, more preferably no more than about 4%, more preferably no more than about 3.9%, more preferably no more than about 3.8%, more preferably no more than about 3.7%, more preferably no more than about 3.6%, more preferably no more than about 3.5%, more preferably no more than about 3.4%, more preferably no more than about 3.3%, more preferably no more than about 3.2%, more preferably no more than about 3.1%, more preferably no more than about 3%, more preferably no more than about 2.9%, more preferably no more than about 2.8%, more preferably no more than about 2.7%, more preferably no more than about 2.6%, more preferably no more than about 2.5%, more preferably no more than about 2.4%, more preferably no more than about 2.3%, more preferably no more than about 2.2%, more preferably no more than about 2.1%, more preferably no more than about 2%, more preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, and most preferably about 0%.

In some non-limiting examples, a concentration Des-Gly9-Oxytocin may be no more than about 9% after a certain period of shelf life, more preferably no more than about 8.9%, more preferably no more than about 8.8%, more preferably no more than about 8.7%, more preferably no more than about 8.6%, more preferably no more than about 8.5%, more preferably no more than about 8.4%, more preferably no more than about 8.3%, more preferably no more than about 8.2%, more preferably no more than about 8.1%, more preferably no more than about 8%, more preferably no more than about 7.9%, more preferably no more than about 7.8%, more preferably no more than about 7.7%, more preferably no more than about 7.6%, more preferably no more than about 7.5%, more preferably no more than about 7.4%, more preferably no more than about 7.3%, more preferably no more than about 7.2%, more preferably no more than about 7.1%, more preferably no more than about 7%, more preferably no more than about 6.9%, more preferably no more than about 6.8%, more preferably no more than about 6.7%, more preferably no more than about 6.6%, more preferably no more than about 6.5%, more preferably no more than about 6.4%, more preferably no more than about 6.3%, more preferably no more than about 6.2%, more preferably no more than about 6.1%, more preferably no more than about 6%, more preferably no more than about 5.9%, more preferably no more than about 5.8%, more preferably no more than about 5.7%, more preferably no more than about 5.6%, more preferably no more than about 5.5%, more preferably no more than about 5.4%, more preferably no more than about 5.3%, more preferably no more than about 5.2%, more preferably no more than about 5.1%, more preferably no more than about 5%, more preferably no more than about 4.9%, more preferably no more than about 4.8%, more preferably no more than about 4.7%, more preferably no more than about 4.6%, more preferably no more than about 4.5%, more preferably no more than about 4.4%, more preferably no more than about 4.3%, more preferably no more than about 4.2%, more preferably no more than about 4.1%, more preferably no more than about 4%, more preferably no more than about 3.9%, more preferably no more than about 3.8%, more preferably no more than about 3.7%, more preferably no more than about 3.6%, more preferably no more than about 3.5%, more preferably no more than about 3.4%, more preferably no more than about 3.3%, more preferably no more than about 3.2%, more preferably no more than about 3.1%, more preferably no more than about 3%, more preferably no more than about 2.9%, more preferably no more than about 2.8%, more preferably no more than about 2.7%, more preferably no more than about 2.6%, more preferably no more than about 2.5%, more preferably no more than about 2.4%, more preferably no more than about 2.3%, more preferably no more than about 2.2%, more preferably no more than about 2.1%, more preferably no more than about 2%, more preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, and most preferably about 0%.

In some non-limiting examples, the concentration Des-Gly9-Oxytocin may be no more than between about 7 and 9% after a certain period of shelf life, optionally no more than between about 7.1 and 8.9%, optionally no more than between about 7.2 and 8.8%, optionally no more than between about 7.3 and 8.7%, optionally no more than between about 7.4 and 8.6%, optionally no more than between about 7.5 and 8.5%, optionally no more than between about 7.6 and 8.4%, optionally no more than between about 7.7 and 8.3%, optionally no more than between about 7.8 and 8.2%, and optionally no more than between about 7.9 and 8.1%.

In some non-limiting examples, a concentration Asp-5-Oxytocin may be no more than about 9% after a certain period of shelf life, more preferably no more than about 8.9%, more preferably no more than about 8.8%, more preferably no more than about 8.7%, more preferably no more than about 8.6%, more preferably no more than about 8.5%, more preferably no more than about 8.4%, more preferably no more than about 8.3%, more preferably no more than about 8.2%, more preferably no more than about 8.1%, more preferably no more than about 8%, more preferably no more than about 7.9%, more preferably no more than about 7.8%, more preferably no more than about 7.7%, more preferably no more than about 7.6%, more preferably no more than about 7.5%, more preferably no more than about 7.4%, more preferably no more than about 7.3%, more preferably no more than about 7.2%, more preferably no more than about 7.1%, more preferably no more than about 7%, more preferably no more than about 6.9%, more preferably no more than about 6.8%, more preferably no more than about 6.7%, more preferably no more than about 6.6%, more preferably no more than about 6.5%, more preferably no more than about 6.4%, more preferably no more than about 6.3%, more preferably no more than about 6.2%, more preferably no more than about 6.1%, more preferably no more than about 6%, more preferably no more than about 5.9%, more preferably no more than about 5.8%, more preferably no more than about 5.7%, more preferably no more than about 5.6%, more preferably no more than about 5.5%, more preferably no more than about 5.4%, more preferably no more than about 5.3%, more preferably no more than about 5.2%, more preferably no more than about 5.1%, more preferably no more than about 5%, more preferably no more than about 4.9%, more preferably no more than about 4.8%, more preferably no more than about 4.7%, more preferably no more than about 4.6%, more preferably no more than about 4.5%, more preferably no more than about 4.4%, more preferably no more than about 4.3%, more preferably no more than about 4.2%, more preferably no more than about 4.1%, more preferably no more than about 4%, more preferably no more than about 3.9%, more preferably no more than about 3.8%, more preferably no more than about 3.7%, more preferably no more than about 3.6%, more preferably no more than about 3.5%, more preferably no more than about 3.4%, more preferably no more than about 3.3%, more preferably no more than about 3.2%, more preferably no more than about 3.1%, more preferably no more than about 3%, more preferably no more than about 2.9%, more preferably no more than about 2.8%, more preferably no more than about 2.7%, more preferably no more than about 2.6%, more preferably no more than about 2.5%, more preferably no more than about 2.4%, more preferably no more than about 2.3%, more preferably no more than about 2.2%, more preferably no more than about 2.1%, more preferably no more than about 2%, more preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, and most preferably about 0%.

In some non-limiting examples, the concentration Asp-5-Oxytocin may be no more than between about 7 and 9% after a certain period of shelf life, optionally no more than between about 7.1 and 8.9%, optionally no more than between about 7.2 and 8.8%, optionally no more than between about 7.3 and 8.7%, optionally no more than between about 7.4 and 8.6%, optionally no more than between about 7.5 and 8.5%, optionally no more than between about 7.6 and 8.4%, optionally no more than between about 7.7 and 8.3%, optionally no more than between about 7.8 and 8.2%, and optionally no more than between about 7.9 and 8.1%.

In some non-limiting examples, a concentration Glu-4-Oxytocin may be no more than about 9% after a certain period of shelf life, more preferably no more than about 8.9%, more preferably no more than about 8.8%, more preferably no more than about 8.7%, more preferably no more than about 8.6%, more preferably no more than about 8.5%, more preferably no more than about 8.4%, more preferably no more than about 8.3%, more preferably no more than about 8.2%, more preferably no more than about 8.1%, more preferably no more than about 8%, more preferably no more than about 7.9%, more preferably no more than about 7.8%, more preferably no more than about 7.7%, more preferably no more than about 7.6%, more preferably no more than about 7.5%, more preferably no more than about 7.4%, more preferably no more than about 7.3%, more preferably no more than about 7.2%, more preferably no more than about 7.1%, more preferably no more than about 7%, more preferably no more than about 6.9%, more preferably no more than about 6.8%, more preferably no more than about 6.7%, more preferably no more than about 6.6%, more preferably no more than about 6.5%, more preferably no more than about 6.4%, more preferably no more than about 6.3%, more preferably no more than about 6.2%, more preferably no more than about 6.1%, more preferably no more than about 6%, more preferably no more than about 5.9%, more preferably no more than about 5.8%, more preferably no more than about 5.7%, more preferably no more than about 5.6%, more preferably no more than about 5.5%, more preferably no more than about 5.4%, more preferably no more than about 5.3%, more preferably no more than about 5.2%, more preferably no more than about 5.1%, more preferably no more than about 5%, more preferably no more than about 4.9%, more preferably no more than about 4.8%, more preferably no more than about 4.7%, more preferably no more than about 4.6%, more preferably no more than about 4.5%, more preferably no more than about 4.4%, more preferably no more than about 4.3%, more preferably no more than about 4.2%, more preferably no more than about 4.1%, more preferably no more than about 4%, more preferably no more than about 3.9%, more preferably no more than about 3.8%, more preferably no more than about 3.7%, more preferably no more than about 3.6%, more preferably no more than about 3.5%, more preferably no more than about 3.4%, more preferably no more than about 3.3%, more preferably no more than about 3.2%, more preferably no more than about 3.1%, more preferably no more than about 3%, more preferably no more than about 2.9%, more preferably no more than about 2.8%, more preferably no more than about 2.7%, more preferably no more than about 2.6%, more preferably no more than about 2.5%, more preferably no more than about 2.4%, more preferably no more than about 2.3%, more preferably no more than about 2.2%, more preferably no more than about 2.1%, more preferably no more than about 2%, more preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, and most preferably about 0%.

In some non-limiting examples, the concentration Glu-4-Oxytocin may be no more than between about 7 and 9% after a certain period of shelf life, optionally no more than between about 7.1 and 8.9%, optionally no more than between about 7.2 and 8.8%, optionally no more than between about 7.3 and 8.7%, optionally no more than between about 7.4 and 8.6%, optionally no more than between about 7.5 and 8.5%, optionally no more than between about 7.6 and 8.4%, optionally no more than between about 7.7 and 8.3%, optionally no more than between about 7.8 and 8.2%, and optionally no more than between about 7.9 and 8.1%.

In some non-limiting examples, a concentration oxytocin trisulfide may be no more than about 9% after a certain period of shelf life, more preferably no more than about 8.9%, more preferably no more than about 8.8%, more preferably no more than about 8.7%, more preferably no more than about 8.6%, more preferably no more than about 8.5%, more preferably no more than about 8.4%, more preferably no more than about 8.3%, more preferably no more than about 8.2%, more preferably no more than about 8.1%, more preferably no more than about 8%, more preferably no more than about 7.9%, more preferably no more than about 7.8%, more preferably no more than about 7.7%, more preferably no more than about 7.6%, more preferably no more than about 7.5%, more preferably no more than about 7.4%, more preferably no more than about 7.3%, more preferably no more than about 7.2%, more preferably no more than about 7.1%, more preferably no more than about 7%, more preferably no more than about 6.9%, more preferably no more than about 6.8%, more preferably no more than about 6.7%, more preferably no more than about 6.6%, more preferably no more than about 6.5%, more preferably no more than about 6.4%, more preferably no more than about 6.3%, more preferably no more than about 6.2%, more preferably no more than about 6.1%, more preferably no more than about 6%, more preferably no more than about 5.9%, more preferably no more than about 5.8%, more preferably no more than about 5.7%, more preferably no more than about 5.6%, more preferably no more than about 5.5%, more preferably no more than about 5.4%, more preferably no more than about 5.3%, more preferably no more than about 5.2%, more preferably no more than about 5.1%, more preferably no more than about 5%, more preferably no more than about 4.9%, more preferably no more than about 4.8%, more preferably no more than about 4.7%, more preferably no more than about 4.6%, more preferably no more than about 4.5%, more preferably no more than about 4.4%, more preferably no more than about 4.3%, more preferably no more than about 4.2%, more preferably no more than about 4.1%, more preferably no more than about 4%, more preferably no more than about 3.9%, more preferably no more than about 3.8%, more preferably no more than about 3.7%, more preferably no more than about 3.6%, more preferably no more than about 3.5%, more preferably no more than about 3.4%, more preferably no more than about 3.3%, more preferably no more than about 3.2%, more preferably no more than about 3.1%, more preferably no more than about 3%, more preferably no more than about 2.9%, more preferably no more than about 2.8%, more preferably no more than about 2.7%, more preferably no more than about 2.6%, more preferably no more than about 2.5%, more preferably no more than about 2.4%, more preferably no more than about 2.3%, more preferably no more than about 2.2%, more preferably no more than about 2.1%, more preferably no more than about 2%, more preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, and most preferably about 0%.

In some non-limiting examples, the concentration oxytocin trisulfide may be no more than between about 7 and 9% after a certain period of shelf life, optionally no more than between about 7.1 and 8.9%, optionally no more than between about 7.2 and 8.8%, optionally no more than between about 7.3 and 8.7%, optionally no more than between about 7.4 and 8.6%, optionally no more than between about 7.5 and 8.5%, optionally no more than between about 7.6 and 8.4%, optionally no more than between about 7.7 and 8.3%, optionally no more than between about 7.8 and 8.2%, and optionally no more than between about 7.9 and 8.1%.

In some non-limiting examples, a concentration N-acetyloxytocin may be no more than about 9% after a certain period of shelf life, more preferably no more than about 8.9%, more preferably no more than about 8.8%, more preferably no more than about 8.7%, more preferably no more than about 8.6%, more preferably no more than about 8.5%, more preferably no more than about 8.4%, more preferably no more than about 8.3%, more preferably no more than about 8.2%, more preferably no more than about 8.1%, more preferably no more than about 8%, more preferably no more than about 7.9%, more preferably no more than about 7.8%, more preferably no more than about 7.7%, more preferably no more than about 7.6%, more preferably no more than about 7.5%, more preferably no more than about 7.4%, more preferably no more than about 7.3%, more preferably no more than about 7.2%, more preferably no more than about 7.1%, more preferably no more than about 7%, more preferably no more than about 6.9%, more preferably no more than about 6.8%, more preferably no more than about 6.7%, more preferably no more than about 6.6%, more preferably no more than about 6.5%, more preferably no more than about 6.4%, more preferably no more than about 6.3%, more preferably no more than about 6.2%, more preferably no more than about 6.1%, more preferably no more than about 6%, more preferably no more than about 5.9%, more preferably no more than about 5.8%, more preferably no more than about 5.7%, more preferably no more than about 5.6%, more preferably no more than about 5.5%, more preferably no more than about 5.4%, more preferably no more than about 5.3%, more preferably no more than about 5.2%, more preferably no more than about 5.1%, more preferably no more than about 5%, more preferably no more than about 4.9%, more preferably no more than about 4.8%, more preferably no more than about 4.7%, more preferably no more than about 4.6%, more preferably no more than about 4.5%, more preferably no more than about 4.4%, more preferably no more than about 4.3%, more preferably no more than about 4.2%, more preferably no more than about 4.1%, more preferably no more than about 4%, more preferably no more than about 3.9%, more preferably no more than about 3.8%, more preferably no more than about 3.7%, more preferably no more than about 3.6%, more preferably no more than about 3.5%, more preferably no more than about 3.4%, more preferably no more than about 3.3%, more preferably no more than about 3.2%, more preferably no more than about 3.1%, more preferably no more than about 3%, more preferably no more than about 2.9%, more preferably no more than about 2.8%, more preferably no more than about 2.7%, more preferably no more than about 2.6%, more preferably no more than about 2.5%, more preferably no more than about 2.4%, more preferably no more than about 2.3%, more preferably no more than about 2.2%, more preferably no more than about 2.1%, more preferably no more than about 2%, more preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, and most preferably about 0%.

In some non-limiting examples, the concentration N-acetyloxytocin may be no more than between about 7 and 9% after a certain period of shelf life, optionally no more than between about 7.1 and 8.9%, optionally no more than between about 7.2 and 8.8%, optionally no more than between about 7.3 and 8.7%, optionally no more than between about 7.4 and 8.6%, optionally no more than between about 7.5 and 8.5%, optionally no more than between about 7.6 and 8.4%, optionally no more than between about 7.7 and 8.3%, optionally no more than between about 7.8 and 8.2%, and optionally no more than between about 7.9 and 8.1%.

In some non-limiting examples, a concentration each unspecified impurity may be no more than about 2% after a certain period of shelf life, more preferably no more than about 1.9%, more preferably no more than about 1.8%, more preferably no more than about 1.7%, more preferably no more than about 1.6%, more preferably no more than about 1.5%, more preferably no more than about 1.4%, more preferably no more than about 1.3%, more preferably no more than about 1.2%, more preferably no more than about 1.1%, more preferably no more than about 1%, more preferably no more than about 0.9%, more preferably no more than about 0.8%, more preferably no more than about 0.7%, more preferably no more than about 0.6%, more preferably no more than about 0.5%, more preferably no more than about 0.4%, more preferably no more than about 0.3%, more preferably no more than about 0.2%, more preferably no more than about 0.1%, and most preferably about 0%.

In some non-limiting examples, the concentration of each unspecified impurity may be no more than between about 0.01 and 2% after a certain period of shelf life, optionally no more than between about 0.1 and 1.9%, optionally no more than between about 0.2 and 1.8%, optionally no more than between about 0.3 and 1.7%, optionally no more than between about 0.4 and 1.6%, optionally no more than between about 0.5 and 1.5%, optionally no more than between about 0.6 and 1.4%, optionally no more than between about 0.7 and 1.3%, optionally no more than between about 0.8 and 1.2%, and optionally no more than between about 0.9 and 1.1%.

According to some aspects, the concentration of impurities present in the composition after a certain period of shelf life may be such that the composition does not undergo a physical change. Examples of physical change include, but are not limited to, color change and/or insoluble particle formation.

Unless indicated otherwise, the percentages of impurities expressed herein are expressed as % (w/w) of the active agent.

The present disclosure is also directed to methods of making the compositions as described herein. In some non-limiting examples, the method may include preparing a bulk solution and sterilizing the bulk solution. The method may further include providing a portion of the sterilized bulk solution in a container as described herein.

In some non-limiting examples, preparing the bulk solution be include providing a vessel having a carrier therein, such as water for injection. The method may further include combining the carrier with one or more of the components described herein

In some examples, the method may include combining the carrier with the tonicity adjusting agent, one or more of the buffer components, one or more of the pH adjusting agents, and the oxytocin and/or pharmaceutically acceptable salt thereof. It should be understood that each of the composition components may independently be combined concurrently with one or more of the other composition components. Additionally or alternatively, each of the composition components may independently be combined sequentially in relation to one or more of the other composition components. In some non-limiting examples, the method may include stirring, mixing, or otherwise agitating the solution prior to, during, or after each composition component is combined therewith.

According to some aspects, the method may further include sterilizing the bulk solution as described herein. The method may further include providing a portion of the sterilized bulk solution in a container as described herein.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

### EXAMPLES

### Example I: Manufacturing Compositions

A bulk solution was prepared according to Table 1 below.

TABLE 1

| Ingredient | Concentration | |
|---|---|---|
| Oxytocin Acetate | 0.06 | IU/mL* |
| Sodium Acetate Trihydrate | 0.2 | mg/mL |
| Acetic acid, Glacial | 0.514 | mg/mL |
| Sodium Chloride | 9.0 | mg/mL |

| | |
|---|---|
| Acetic acid, Glacial | Q.S. to pH 3.0 to 5.0 |
| Sodium Hydroxide | Q.S. to pH 3.0 to 5.0 |
| Water For Injection | Q.S. |

Note:
*With 7% overages.
Q.S.: Quantity Sufficient

To prepare the bulk solution, first, sufficient water for injection for a total batch size was collected in the manufacturing vessel. Then, the sodium chloride was added and dissolved, and the combination was stirred until a clear solution was obtained. The sodium acetate trihydrate was then added and dissolved, and the combination was again stirred until a clear solution was obtained. The acetic acid was then added and dissolved, and the combination was again stirred until a clear solution was obtained. The pH of the solution was then checked to confirm a pH of between about 3.0 and 5.0. If the pH was not between this range, it was adjusted with a solution of the sodium hydroxide and acetic acid. The oxytocin acetate was then added and dissolved, and the combination was again stirred until a clear solution was obtained. The volume was then made to 100% using water for injection, and the composition was stirred to provide the bulk solution.

The bulk solution was then filtered through a 0.2 m filter. The sterilized bulk solution was then filled into containers to provide the presentations shown in Table 2.

TABLE 2

| | Amount of Composition | |
|---|---|---|
| Presentation | in Container | Container Type |
| 1 | 125 mL | Clear glass vial stoppered with |
| | | a rubber stopper and sealed |
| | | with aluminum flip off seal |
| 2 | 250 mL | Clear glass vial stoppered with |
| | | a rubber stopper and sealed |
| | | with aluminum flip off seal |
| 3 | 500 mL | IV bag, sealed with twist off |
| | | port and packed into |
| | | aluminum pouch |

A second bulk solution was prepared according to Table 3 below.

TABLE 3

| Ingredient | Concentration | |
|---|---|---|
| Oxytocin Acetate | 0.02 | IU/mL* |
| Sodium Acetate Trihydrate | 0.2 | mg/mL |
| Acetic acid, Glacial | 0.514 | mg/mL |
| Sodium Chloride | 9.0 | mg/mL |

| | |
|---|---|
| Acetic acid, Glacial | Q.S. to pH 3.0 to 5.0 |
| Sodium Hydroxide | Q.S. to pH 3.0 to 5.0 |
| Water For Injection | Q.S. |

Note:
*With 7% overages.
Q.S.: Quantity Sufficient

To prepare the bulk solution, first, sufficient water for injection for a total batch size was collected in the manufacturing vessel. Then, the sodium chloride was added and dissolved, and the combination was stirred until a clear solution was obtained. The sodium acetate trihydrate was then added and dissolved, and the combination was again stirred until a clear solution was obtained. The acetic acid was then added and dissolved, and the combination was again stirred until a clear solution was obtained. The pH of the solution was then checked to confirm a pH of between about 3.0 and 5.0. If the pH was not between this range, it was adjusted with a solution of the sodium hydroxide and acetic acid. The oxytocin acetate was then added and dissolved, and the combination was again stirred until a clear solution was obtained. The volume was then made to 100% using water for injection, and the composition was stirred to provide the bulk solution.

The bulk solution was then filtered through a 0.2 m filter. Then, 1,000 mL aliquots of the sterilized bulk solution was filled into IV bags, sealed with twist off port and packed into aluminum pouch.

### Example II: Stability

The stability of presentations 1 and 2 of Table 2 were determined after 6 months of storage, either upright or inverted, at 5° C., 25° C., and 40° C. To determine stability, initial measurements for pH, assay concentration, and impurities were determined. The measurements were taken again at one month, two months, three, months, optionally four months and 25 days, and six months.

The impurities identified included Des-Gly9-Oxytocin, Asp-5-Oxytocin, Glu-4-Oxytocin, oxytocin trisulfide, and N-acetyloxytocin. The impurities levels were determined using a reversed-phase gradient HPLC method with UV detection. A HPLC column with a C-18 stationary phase was used. The mobile phases are as shown in Table 4. Table 4 also shows the diluent solution used in the analysis.

TABLE 4
Solutions for Gradient HPLC Method

| Solution | Preparation |
|---|---|
| Mobile Phase A | Weigh about 5.52 g of Sodium Phosphate |
| | Monobasic Monohydrate, and dissolve in |
| | 2000 mL of Water. Adjust pH to 2.70 ± 0.05 |
| | with Phosphoric Acid. Mix well. |
| | Filter through a conditioned C18 extraction |
| | disk under vacuum |
| Mobile Phase B | Mix 1000 mL of Acetonitrile and 1000 mL of |
| | Purified Water. Degas by sonicating under |
| | vacuum for 2 minutes. |
| Diluent | Thoroughly mix 5.0 mL of Glacial Acetic |
| | Acid and 2000 mL of Water. Filter through a |
| | conditioned C18 extraction disk under |
| | vacuum. |

Table 5 shows the standards used in the analysis.

TABLE 5
Standards for Gradient HPLC Method

| Solution | Preparation |
|---|---|
| Stock Standard | Quantitatively transfer the contents of 1 vial |
| Solution (SS) | of Oxytocin USP Reference Standard with |
| | Diluent into a volumetric flask to obtain an |
| | oxytocin concentration of approximately 10 |
| | unit/mL |
| Intermediate | Pipet 3.5 mL of Oxytocin Stock Standard |
| Standard Solution | Solution into a 25-mL volumetric flask. Dilute |
| (IS) | to volume with Diluent. Mix well. |
| Working Standard | Pipet 4.0 mL of Oxytocin Intermediate |
| Solution (WS) | Standard Solution into a 100-mL volumetric |
| | flask. Dilute to volume with Diluent. Mix well. |
| Check Standard | Prepare a second Oxytocin Stock Standard |
| Solutions (SCS) | Solution, Intermediate Standard Solution, and |
| | Working Standard Solution using the same lot of |
| | Oxytocin USP Reference Standard. |
| Sensitivity | Pipet 1.7 mL of Oxytocin Working Standard |
| Solution | Solution into a 100-mL volumetric flask. |
| | Dilute to volume with Diluent. Mix well. |
| Impurity Stock | Weigh about 2.5 mg of each impurity (Des- |
| Solutions | Gly9-Oxytocin, Asp-5-Oxytocin, Glu-4- |
| | Oxytocin, oxytocin trisulfide, and N- |
| | acetyloxytocin) into individual 25-mL |
| | volumetric flasks, and dissolve and dilute to |
| | volume with Diluent. Mix well. |
| Mixed Impurity | Pipet 1.0 mL of Impurity Stock Solution of |
| Solution | each impurity (Des-Gly9-Oxytocin, Asp-5- |
| | Oxytocin, Glu-4-Oxytocin, oxytocin |
| | trisulfide, and N-acetyloxytocin) into a 100- |
| | mL volumetric flask. Dilute to volume with |
| | Diluent. Mix well. |
| Resolution Solution | Pipet 4.0 mL of Oxytocin Intermediate |
| (Marker Solution | Standard Solution and 1.0 mL of Mixed |
| | Impurity Solution into a 100-mL volumetric |
| | flask and dilute to volume with Diluent. Mix |
| | well. |
| Sample Preparation | None, inject as is. |

The chromatographic conditions used for the analysis are shown in Tables 6 and 7.

TABLE 6
Liquid Chromatography System and Chromatographic Conditions

| System | HPLC or equivalent | |
|---|---|---|
| Column | Agilent InfinityLab Poroshell 120 | |
| | EC-C18, 2.7 μm, 3.0 × 150 mm | |
| Column Temperature | 35° | C. |
| Sample Temperature | 5° | C. |
| Injection Volume | 900 | μL |
| Draw/Eject speed | 500 | μL/min |
| Flow Rate | 0.45 | mL/min |

| Detection | VWD 214 nm, for ID by retention time, | | |
|---|---|---|---|
| | Assay and Impurities. | | |
| | DAD: 214 nm, Bandwidth - 4 nm; Reference - | | |
| | OFF, for ID by UV. Spectrum is collected | | |
| | from 190 nm to 400 nm | | |
| Mobile Phase A | pH 2.70 sodium phosphate monobasic | | |
| Mobile Phase B | Acetonitrile:water (1:1) | | |
| | Time (min) | % A | % B |
| Gradient | 0.00 | 80 | 20 |
| | 3.50 | 80 | 20 |
| | 36.50 | 58 | 42 |
| | 46.00 | 30 | 70 |
| | 48.00 | 30 | 70 |
| | 48.10 | 80 | 20 |
| | 65.00 | 80 | 20 |

TABLE 7
Sample Sequence

| Number of Injections | Sample Name |
|---|---|
| At least 2 (in | Diluent |
| individual vials) | |
| 1 | Sensitivity Solution |
| 1 | Resolution Solution |
| 6 (in individual vials) | Working Standard Solution (WS) |
| 1 | Check Working Standard Solution (CS) |
| 1 | Working Standard Solution (WS) |
| Up to 10 | Samples |
| 1 | Bracketing Standard (WS) |

The system suitability requirements were as follows:

In the last injection Diluent, no significant peaks are at the retention times of Oxytocin and Identified Impurities. A significant peak is defined as a peak which has a peak area ≥15% of Oxytocin peak area in the Sensitivity Solution injection.

In the Sensitivity Solution the signal-to-noise ratio (S/N) of the Oxytocin peak is ≥70.

In the Resolution Solution:

The resolution between Oxytocin and Gly9-OX is ≥2.5

The resolution between Gly9-OX and Asp5-OX is ≥1.5

The resolution between Asp5-OX and Glu4-OX is ≥1.8

The resolution between Glu4-OX and Trisulfide-OX is ≥1.8

The % RSD of 6 replicate injections of the Oxytocin Working Standard is ≤2.0%

The tailing factor (T) of the Oxytocin peak in the first Working Standard injection is 0.8≤T≤1.4

The Check Standard conformity must be within the range 97-103%.

$$Standard Conformity = \frac{{A}_{ck std}}{{A}_{avg std}} \times \frac{{C}_{std}}{{C}_{ck std}} \times 100 \%$$

where A_{ck std} is the peak area of Oxytocin in Oxytocin Check Working Standard Solution injection, A_{avg std} is the average peak area of Oxytocin in 6 initial Oxytocin Working Standard Solution injections, C_{std} is the concentration of Oxytocin in Oxytocin Stock Standard Solution, in unit/mL, and C_{ck std} is the concentration of Oxytocin in Oxytocin Check Stock Standard Solution, in unit/mL.

The Oxytocin peak area in each ongoing Oxytocin Working Standard Solution injection must be 98.0% to 102.0% of the average peak area of Oxytocin in system suitability injections (n=6)

Assay quantitation was accomplished by comparing corresponding Oxytocin peak areas from a Sample solution chromatogram to that of the average area of sample bracketing external Reference Standard solution of known concentration.

Impurities are calculated as a percentage of area of the Oxytocin peak present in the sample.

Table 8 shows the Relative Retention Time (RRT) for several unspecified related substances, also referred to as unknown impurities. In a preferred embodiment, the total amount of these unknown impurities is not more than 1%.

TABLE 8
Unknown Impurities in Oxytocin Acetate Compositions

| Impurity | Approximate RRT |
|---|---|
| A | 0.37 |
| B | 0.40 |
| C | 0.54 |
| D | 0.60 |
| E | 0.67 |
| F | 0.70 |
| G | 0.71 |
| H | 0.88 |
| I | 0.91 |
| J | 1.03 |
| K | 1.26 |
| L | 1.27 |
| M | 1.30 |
| N | 1.32 |
| O | 1.38 |
| P | 1.52 |
| Q | 1.56 |
| R | 1.62 |

The results of this study for Presentations 1 and 2 are shown in Tables 9 and 10 below, respectively.

TABLE 9
Presentation 1

| | | | | | | | Impurity (% of API Peak) | | | | | | | | | |
|---|---|---|---|---|---|---|---|---|---|---|---|---|---|---|---|---|
| | | | | | Known | | | | | Unknown | | | | | | |
| | Time | | Assay | Assay | Gly | Asp | Glu | | | RRT | RRT | RRT | RRT | RRT | RRT | RRT |
| Condition | Point | pH | mU/mL | in % | 9 | 5 | 4 | Trisulfide | Acetyl | 0.37 | 0.40 | 0.54 | 0.60 | 0.67 | 0.70 | 0.71 |
| Tentative | | 3.0 to | | 90 to | NMT | NMT | NMT | NMT | NMT | NMT | | | | | | |
| Specifications | | 5.0 | | 110 | 8% | 8% | 8% | 8% | 8% | 1.0% | | | | | | |
| Sample 1 | Initial | 4.03 | 62.76 | 104.60 | ND | ND | ND | ND | 0.43 | — | — | — | — | — | — | — |
| Sample 2 | | 4.06 | 62.62 | 104.37 | ND | ND | ND | ND | 0.41 | — | — | — | — | — | — | — |
| 5° C. | 1 M | 4.10 | 63.35 | 105.58 | ND | ND | ND | ND | 0.43 | — | — | — | — | — | — | — |
| UPRIGHT | 2 M | 4.09 | 62.95 | 104.91 | ND | ND | ND | ND | 0.48 | — | — | — | — | — | — | — |
| | 3 M | 4.06 | 63.51 | 105.85 | ND | ND | ND | ND | 0.4 [symbol] | — | — | — | — | — | — | — |
| | 6 M | 4.09 | 62.08 | 103.47 | ND | ND | ND | ND | 0.43 | — | — | — | — | — | — | — |
| 5° C. | 1 M | 4.11 | 63.64 | 106.06 | ND | ND | ND | ND | 0.42 | — | — | — | — | — | — | — |
| INVERT | 2 M | 4.10 | 62.46 | 104.10 | ND | ND | ND | ND | 0.47 | — | — | — | — | — | — | — |
| | 3 M | 4.07 | 63.41 | 105. [symbol] | ND | ND | ND | ND | 0.47 | — | — | — | — | — | — | — |
| | 6 M | 4.06 | [symbol] 2.02 | 103.37 | ND | ND | ND | ND | 0.43 | — | — | — | — | — | — | — |
| 25° C. | 1 M | 4.11 | 63.12 | 105.20 | ND | ND | 0.17 | ND | 0.45 | — | — | — | — | — | 0.61 | — |
| UPRIGHT | 2 M | 4.10 | 62.20 | 103.67 | 0.29 | ND | 0.23 | ND | 0.47 | — | — | — | — | — | 0.16 | — |
| | 3 M | 4.06 | [symbol] 2.16 | 103.60 | 0.42 | ND | 0.34 | ND | 0.43 | — | — | — | — | — | — | — |
| | 6 M | 4.10 | 59.67 | 99.45 | 0.72 | 0.20 | 0. [symbol] 3 | ND | 0.43 | — | — | — | — | — | — | — |
| 25° C. | 1 M | 4.11 | 62.85 | 104.77 | ND | ND | ND | ND | 0.45 | — | — | — | — | — | 0.36 | — |
| INVERT | 2 M | 4.10 | 62.11 | 103.52 | 0.30 | ND | 0.28 | ND | 0.48 | — | — | — | — | — | — | — |
| | 3 M | 4.07 | 60.45 | 100.75 | 0.40 | ND | 0.36 | ND | 0.45 | — | — | — | — | — | — | — |
| | 4 M 25 D | 4.05 | 59.68 | 99.47 | 0.49 | 0.15 | 0.51 | ND | 0.42 | — | — | — | — | — | — | — |
| | 6 M | 4.09 | 59.25 | [symbol] 9.75 | 0.70 | 0.19 | 0.66 | ND | 0.44 | — | — | — | — | — | — | — |
| 40° C. | 1 M | 4.11 | [symbol] 0.75 | 101.25 | 0.61 | 0.18 | 0. [symbol] 0 | ND | 0.51 | — | — | — | — | — | 1.07 | — |
| UPRIGHT | 2 M | 4.10 | 57.78 | 86.29 | 1.12 | 0.28 | 1.08 | ND | 0.47 | — | — | — | — | — | 0.16 | — |
| | 3 M | 4.08 | 56.10 | 93.50 | 1.95 | 0.43 | 1.60 | 0.20 | 0.4 [symbol] | — | — | — | — | — | — | — |
| | 6 M | 4.08 | 49.50 | 82.50 | 4.83 | 1.20 | 3.10 | 0.27 | 0.52 | — | — | — | — | — | — | — |
| 40° C. | 1 M | 4.13 | 60. [symbol] 5 | 101.08 | 0.69 | 0.21 | 0. [symbol] 4 | ND | 0.47 | — | — | — | — | — | 0.20 | 0.19 |
| INVERT | 2 M | 4.11 | 57.39 | 95.65 | 1.17 | 0.31 | 1.06 | ND | 0.55 | — | — | — | — | — | — | — |
| | 3 M | 4.08 | 55.39 | 92.32 | 1.82 | 0.48 | 1.63 | 0.33 | 0.53 | — | — | — | — | — | — | — |
| | 4 M 25 D | 4.10 | 51.62 | 86.03 | 2.72 | 0. [symbol] | 2.42 | 0.28 | 0.52 | — | — | — | 0.20 | — | — | — |
| | 6 M | 4.08 | 49.33 | 82.22 | 3.79 | 0.89 | 3.18 | 0.48 | 0.65 | 0.19 | 0.19 | — | — | — | — | — |

| | | Impurity (% of API Peak) | | | | | | | | | | | | |
|---|---|---|---|---|---|---|---|---|---|---|---|---|---|---|
| | | Unknown | | | | | | | | | | Total | Total | |
| | Time | RRT | RRT | RRT | RRT | RRT | RRT | RRT | RRT | RRT | RRT | Know | Unknown | Total |
| Condition | Point | 0.88 | 1.03 | 1.26 | 1.27 | 1.30 | 1.32 | 1.38 | 1.52 | 1.56 | 1.62 | Impurities | impurities | impurities |
| Tentative | | | | | | | | | | | | | | NMT |
| Specifications | | | | | | | | | | | | | | 16% |
| Sample 1 | Initial | — | — | — | — | — | 0.10 | 0.49 | — | — | — | 0.43 | 0.60 | 1.03 |
| Sample 2 | | — | — | — | — | — | 0.10 | 0.51 | — | — | — | 0.41 | 0.61 | 1.02 |
| 5° C. | 1 M | — | — | — | — | — | 0.29 | 0.50 | — | — | — | 0.43 | 0.78 | 1.22 |
| UPRIGHT | 2 M | — | — | — | — | — | — | 0.57 | — | — | — | 0.48 | 0.57 | 1.05 |
| | 3 M | — | — | — | — | — | 0.31 | 0.51 | — | — | — | 0.4 [symbol] | 0.82 | 1.28 |
| | 6 M | — | — | — | — | — | 0.30 | 0.59 | — | — | — | 0.43 | 0.89 | 1.32 |
| 5° C. | 1 M | — | — | — | — | — | 0.25 | 0.52 | — | — | — | 0.42 | 0.77 | 1.1 [symbol] |
| INVERT | 2 M | — | — | — | 0.15 | — | — | 0.52 | — | — | — | 0.47 | 0.67 | 1.14 |
| | 3 M | — | — | — | — | — | 0.31 | 0.53 | — | — | — | 0.47 | 0.84 | 1.31 |
| | 6 M | — | — | — | — | — | 0.33 | 0.54 | — | — | — | 0.43 | 0.87 | 1.30 |
| 25° C. | 1 M | — | — | — | — | — | 0.35 | 0.52 | — | — | — | 0.62 | 1.48 | 2.10 |
| UPRIGHT | 2 M | — | — | — | — | — | — | 0.53 | — | — | — | 0.99 | 0.69 | 1.68 |
| | 3 M | — | — | — | — | — | 0.47 | 0.57 | — | — | — | 1.19 | 1.04 | 2.23 |
| | 6 M | — | — | — | — | — | 0.57 | 0.5 [symbol] | — | — | — | 1.98 | 1.13 | 3.11 |
| 25° C. | 1 M | — | — | — | — | — | 0.39 | 0.50 | — | — | — | 0.45 | 1.25 | 1.69 |
| INVERT | 2 M | — | — | — | — | — | — | 0.53 | — | — | — | 1.06 | 0.53 | 1.59 |
| | 3 M | — | — | — | — | — | 0.85 | 0.56 | — | — | — | 1.21 | 1.41 | 2.62 |
| | 4 M 25 D | — | — | — | — | 0.04 | 0.80 | 0.54 | — | — | — | 1.57 | 1.38 | 2.95 |
| | 6 M | — | — | — | — | — | 0.65 | 0.56 | — | — | — | 1.99 | 1.21 | 3.20 |
| 40° C. | 1 M | — | — | — | — | — | 0.39 | 0.55 | — | 0.33 | — | 1.89 | 2.33 | 4.22 |
| UPRIGHT | 2 M | — | — | — | — | — | — | 0.54 | — | — | — | 2.95 | 0.70 | 3.65 |
| | 3 M | — | — | — | — | — | 0.37 | 0.53 | — | — | — | 4.65 | 0.90 | 5.54 |
| | 6 M | — | 0.76 | — | — | — | 0.40 | 0.61 | — | 0.29 | 0.55 | 9.92 | 2.61 | 12.53 |
| 40° C. | 1 M | 0.20 | — | — | — | — | 0.36 | 0.53 | — | — | — | 2.03 | 1.48 | 3.49 |
| INVERT | 2 M | — | — | — | — | — | 0.46 | 0.53 | — | — | — | 3.09 | 0.99 | 4.08 |
| | 3 M | — | — | — | 0.29 | — | 0. [symbol] 6 | 0.59 | — | — | — | 4.79 | 1.54 | 6.33 |
| | 4 M 25 D | — | — | — | — | 0.11 | 0.07 | 0.56 | — | — | — | 6.60 | 0.94 | 7.54 |
| | 6 M | — | 0.40 | 0.19 | 0.21 | — | — | 0.52 | — | 0.25 | 0.35 | 8.97 | 2.30 | 11.27 |

[symbol]

indicates data missing or illegible when filed

TABLE 10
Presentation 2

| | | | | | Impurity (% of API Peak) | | | | | | | | |
|---|---|---|---|---|---|---|---|---|---|---|---|---|---|
| | | | | | Known | | | | | Unknown | | | |
| | Time | | Assay | Assay | Gly | Asp | Glu | | | RRT | RRT | RRT | RRT |
| Condition | Point | pH | mU/mL | in % | 9 | 5 | 4 | Trisulfide | Acetyl | 0.54 | 0.67 | 0.70 | 0.71 |
| Tentative | | 3.0 to | | 90 to | NMT | NMT | NMT | NMT | NMT | | NMT | | |
| Specifications | | 5.0 | | 110 | 8% | 8% | 8% | 8% | 8% | | 1.0% | | |
| Sample 1 | Initial | 4.11 | 64.39 | 107.32 | ND | ND | ND | NO | 0.44 | 0.24 | — | — | — |
| Sample 2 | | 4.10 | 64.58 | 107. [symbol] 0 | ND | ND | ND | ND | 0.42 | — | — | — | 0.72 |
| 5° C. | 1 M | 4.16 | [symbol] 5.29 | 108.82 | ND | ND | ND | ND | 0.47 | — | 0.17 | — | — |
| UPRIGHT | 2 M | 4.12 | 64.55 | 107.58 | ND | ND | ND | ND | 0.47 | — | — | — | — |
| | 3 M | 4.10 | [symbol] 5.36 | 108.93 | ND | ND | ND | ND | 0.41 | — | — | — | — |
| | 6 M | 4.10 | 64.06 | 106.77 | ND | ND | ND | ND | 0.43 | — | — | — | — |
| 5° C. | 1 M | 4.14 | [symbol] 5.37 | 108.95 | ND | ND | ND | ND | 0.44 | — | — | 0.38 | — |
| INVERT | 2 M | 4.13 | 64.67 | 107.78 | ND | ND | ND | ND | 0.47 | — | — | — | — |
| | 3 M | 4.10 | [symbol] 5.4 [symbol] | 109.10 | ND | ND | ND | ND | 0.47 | — | — | — | — |
| | 6 M | 4.10 | 63.94 | 106.57 | ND | ND | ND | ND | 0.43 | — | — | — | — |
| 25° C. | 1 M | 4.14 | 64.81 | 108.02 | ND | ND | ND | ND | 0.47 | — | — | — | — |
| UPRIGHT | 2 M | 4.14 | 63.94 | 10 [symbol] .57 | 0.25 | ND | 0.32 | ND | 0.44 | — | — | — | — |
| | 3 M | 4.07 | 63.98 | 10 [symbol] . [symbol] 3 | 0.3 [symbol] | ND | 0.32 | ND | 0.46 | — | — | — | — |
| | 6 M | 4.11 | 61.56 | 102.60 | 0.66 | 0.19 | 0.59 | ND | 0.46 | — | — | — | — |
| 25° C. | 1 M | 4.14 | 64.90 | 108.17 | ND | ND | ND | ND | 0.42 | — | — | — | — |
| INVERT | 2 M | 4.14 | [symbol] 3.56 | 105.93 | 0.25 | ND | 0.22 | ND | 0.46 | — | 0.15 | — | — |
| | 3 M | 4.14 | 64.03 | 106.72 | 0.32 | ND | 0.32 | ND | 0.42 | — | — | — | — |
| | 4 M 25 D | 4.12 | [symbol] 1.89 | 103.15 | 0.51 | 0.18 | 0.52 | ND | 0.42 | — | — | — | — |
| | 6 M | 4.11 | 61.32 | 102.20 | 0.59 | 0.19 | 0.61 | ND | 0.44 | — | — | — | — |
| 40° C. | 1 M | 4.14 | 62.03 | 103.39 | 0.55 | ND | 0. [symbol] 9 | ND | 0.47 | — | — | — | — |
| UPRIGHT | 2 M | 4.14 | 59.08 | 98.43 | 1.11 | 0.30 | 1.00 | ND | 0.53 | — | — | 0.18 | — |
| | 3 M | 4.12 | 57.0 [symbol] | 95.10 | 1. [symbol] | 0.45 | 1.57 | ND | 0.46 | — | — | — | — |
| | 6 M | 4.11 | 51.08 | 85.13 | 3.59 | 0.85 | 2.93 | 0.22 | 0.61 | — | — | — | — |
| 40° C. | 1 M | 4.15 | 62.19 | 103.64 | 0.57 | 0.19 | 0.58 | ND | 0.46 | — | — | — | — |
| INVERT | 2 M | 4.15 | 59.3 [symbol] | 98.93 | 1.09 | 0.31 | 0.98 | ND | 0.50 | — | — | — | — |
| | 3 M | 4.12 | 57.20 | 95.33 | 1.60 | 0.44 | 1.49 | ND | 0.52 | — | — | — | — |
| | 4 M 25 D | 4.11 | 53.15 | 88.56 | 2.54 | 0.63 | 2.33 | ND | 0.48 | — | — | — | — |
| | 6 M | 4.09 | 50.83 | 84.72 | 3.55 | 0.88 | 3.00 | 0.24 | 0.54 | — | — | — | — |

| | | Impurity (% of API Peak) | | | | | | | | | | |
|---|---|---|---|---|---|---|---|---|---|---|---|---|
| | | Unknown | | | | | | | | Total | Total | |
| | Time | RRT | RRT | RRT | RRT | RRT | RRT | RRT | RRT | Known | Unknown | Total |
| Condition | Point | 0.91 | 1.03 | 1.27 | 1.32 | 1.38 | 1.52 | 1.56 | 1.62 | Impurities | impurities | impurities |
| Tentative | | | | | | | | | | | | NMT |
| Specifications | | | | | | | | | | | | 16% |
| Sample 1 | Initial | — | — | — | 0.05 | — | — | — | — | 0.44 | 0.29 | 0.73 |
| Sample 2 | | — | — | — | — | — | — | — | — | 0.42 | 0.72 | 1.14 |
| 5° C. | 1 M | — | — | — | 0.20 | 0.29 | — | — | — | 0.47 | 0.66 | 1.13 |
| UPRIGHT | 2 M | — | — | — | 0.27 | 0.30 | — | — | — | 0.47 | 0.57 | 1.04 |
| | 3 M | — | — | — | 0.28 | 0.25 | — | — | — | 0.41 | 0.53 | 0.94 |
| | 6 M | — | — | — | 0.30 | 0.27 | — | — | — | 0.43 | 0.57 | 1.00 |
| 5° C. | 1 M | — | — | — | 0.27 | 0.28 | — | — | — | 0.44 | 0.92 | 1.3 [symbol] |
| INVERT | 2 M | — | — | 0.16 | 0.23 | 0.24 | — | — | — | 0.47 | 0.63 | 1.10 |
| | 3 M | — | — | 0.18 | 0.27 | 0.23 | — | — | — | 0.47 | 0.68 | 1.15 |
| | 6 M | — | — | 0.16 | 0.33 | 0.29 | — | — | — | 0.43 | 0.78 | 1.21 |
| 25° C. | 1 M | 0.1 [symbol] | — | — | 0.3 [symbol] | 0.25 | — | — | — | 0.47 | 0.80 | 1.26 |
| UPRIGHT | 2 M | — | — | 0.15 | 0.38 | 0.28 | — | — | — | 1.01 | 0.79 | 1.80 |
| | 3 M | — | — | — | 0.38 | 0.24 | — | — | — | 1.14 | 0.62 | 1.76 |
| | 6 M | — | — | 0.16 | 0.60 | 0.28 | — | — | — | 1.90 | 1.04 | 2.94 |
| 25° C. | 1 M | — | — | — | 0.29 | 0.24 | — | — | — | 0.42 | 0.53 | 0.95 |
| INVERT | 2 M | — | — | — | 0.41 | 0.29 | — | — | — | 0.93 | 0.85 | 1.78 |
| | 3 M | — | — | — | 0.38 | 0.25 | — | — | — | 1.06 | 0.63 | 1.69 |
| | 4 M 25 D | — | — | 0.17 | 0.65 | 0.30 | — | — | — | 1.61 | 1.12 | 2.73 |
| | 6 M | — | — | — | 0.61 | 0.33 | — | — | — | 1.83 | 0.94 | 2.77 |
| 40° C. | 1 M | — | — | — | 0.28 | 0.28 | — | — | — | 1.61 | 0.56 | 2.17 |
| UPRIGHT | 2 M | — | — | — | 0.44 | 0.29 | — | — | — | 2.94 | 0.91 | 3.85 |
| | 3 M | — | — | 0.20 | 0.41 | 0.28 | — | — | — | 4.16 | 0.89 | 5.05 |
| | 6 M | — | 0.34 | 0.19 | 0.36 | 0.31 | — | — | 0.78 | 8.20 | 1.98 | 10.18 |
| 40° C. | 1 M | — | — | 0.24 | 0.37 | 0.34 | — | — | — | 1.80 | 0.94 | 2.74 |
| INVERT | 2 M | — | — | 0.16 | 0.37 | 0.28 | 0.49 | — | — | 2.88 | 4.30 | 4.18 |
| | 3 M | — | — | — | 0.42 | 0.26 | — | — | — | 4.05 | 0.68 | 4.73 |
| | 4 M 25 D | — | — | — | — | 0.30 | — | 0.21 | — | 5.98 | 0.51 | 6.49 |
| | 6 M | — | 0.31 | 0.25 | 0.43 | 0.30 | — | — | 0. [symbol] 0 | 8.21 | 2.09 | 10.30 |

[symbol]

indicates data missing or illegible when filed

What is claimed is:

1. A stable oxytocin composition comprising:
between about 0.04 and 0.08 IU/mL of oxytocin and/or a pharmaceutically acceptable salt thereof,
a buffer comprising sodium acetate trihydrate and acetic acid,
a tonicity adjusting agent comprising sodium chloride,
a pH adjusting agent comprising acetic acid, sodium hydroxide, or a combination thereof, and
water,
wherein the composition has a total impurities concentration of equal to or less than 16% after storage of at least six months at 25° C.

2. The composition of claim 1, wherein the oxytocin and/or a pharmaceutically acceptable salt thereof is present in a concentration of about 0.06 IU/mL.

3. The composition of claim 1, wherein the oxytocin and/or a pharmaceutically acceptable salt thereof is present in a concentration of about 0.02 IU/mL.

4. The composition of claim 1, wherein the sodium acetate trihydrate is provided in a concentration of between about 0.1 and 0.3 mg/mL.

5. The composition of claim 4, wherein the sodium acetate trihydrate is provided in a concentration of about 0.2 mg/mL.

6. The composition of claim 1, wherein the acetic acid is provided in a concentration of between about 0.4 and 0.6 mg/mL.

7. The composition of claim 6, wherein the acetic acid is provided in a concentration of between about 0.514 mg/mL.

8. The composition of claim 1, wherein the tonicity adjusting agent is provided I between about 8 and 10 mg/mL.

9. The composition of claim 8, wherein the tonicity adjusting agent is provided I between about 9 mg/mL.

10. The composition of claim 1, wherein the composition has an initial pH range of between about 3.0 to 5.0.

11. The composition of claim 1, wherein the composition has a total impurities concentration of equal to or less than 10% after storage of at least six months at 25° C.

12. A pharmaceutical product comprising a single-use container containing the composition of claim 1.

13. The product of claim 12, wherein the single-use container is an infusion bag or a vial.

14. The product of claim 12, wherein the single-use container contains about 125 mL of the composition.

15. The product of claim 12, wherein the single-use container contains about 250 mL of the composition.

16. The product of claim 12, wherein the single-use container contains about 500 mL of the composition.

17. The product of claim 12, wherein the single-use container contains about 1,000 mL of the composition.

18. A method of making an oxytocin composition comprising:
preparing a bulk solution comprising:
between about 0.04 and 0.08 IU/mL of oxytocin and/or a pharmaceutically acceptable salt thereof,
a buffer comprising sodium acetate trihydrate and acetic acid,
a tonicity adjusting agent comprising sodium chloride,
a pH adjusting agent comprising acetic acid, sodium hydroxide, or a combination thereof, and
water; and
sterilizing the bulk solution.

19. The method of claim 18, wherein the oxytocin and/or a pharmaceutically acceptable salt thereof is present in a concentration of about 0.06 IU/mL.

20. The method of claim 18, wherein the oxytocin and/or a pharmaceutically acceptable salt thereof is present in a concentration of about 0.02 IU/mL.
